(19) United States
(12) Patent Application Publication
(10) Pub. No.: US 2011/0077996 A1
(43) Pub. Date: Mar. 31, 2011

(54) Multimodal Affective-Cognitive Product Evaluation

(76) Inventors: Hyungil Ahn, Cambridge, MA (US); Rosalind Picard, Newton, MA (US)

(21) Appl. No.: 12/567,115

(22) Filed: Sep. 25, 2009

(51) Int. Cl. G06Q 10/00; A61B 5/04

(52) U.S. Cl. 705/10; 600/546

(57) ABSTRACT

Repeated random-outcome trials together with affective, cognitive, and behavioral measures of liking and wanting may be used to assess consumer preferences. In an exemplary implementation of this invention, in each trial, a participant selects one of two sources (e.g., one of two beverage dispensers) of a product (e.g., a beverage). Each source dispenses the product randomly, with a probability initially unknown to the participant, but which he or she may guess while trying to select the most desired product. Affective measures of a participant's facial valence and sympathetic nervous system activation are taken while deciding on, anticipating the arrival of, receiving, using, evaluating, and reflecting on the product. The affective measures are combined with cognitive self-report questionnaire items and with behavioral measures to infer wanting and liking of a product.

### FIELD OF THE INVENTION

This invention relates generally to consumer preference research.

### BACKGROUND OF THE INVENTION

Companies want new products to be successful in the marketplace; however, current evaluation methods do not accurately predict customer decisions and preferences in the marketplace.

In a traditional approach to determining consumer preferences regarding a product, participants experience a product and then only cognitive measures of the participants' experiences (i.e., self-reports) are taken. Cognitive self-report items on questionnaires do not give reliable indications of marketplace outcomes. Nor do focus groups. Large amounts of time, money, and other resources are wasted because of the poor predictions made by these methods.

### SUMMARY OF THE INVENTION

According to principles of this invention, multi-modal measures may be used, rather than cognitive measures only. This multi-modal approach includes affective measures (e.g. sensor measures of facial expression), behavioral measures (e.g., physical number of choices, amount consumed), and cognitive measures (e.g., self-reports). Using this multi-modal approach, data is collected on participants' experiences during anticipatory, decision-making and evaluation processes.

This multi-modal measurement can provide more robust assessment of participants' preferences than cognitive measures alone because of the following main reasons: First, the human brain uses both emotion (affect) and cognition in decision-making and evaluation processes. Second, participants in an experiment are likely to cognitively bias their self-report of what they like. Third, when people are cognitively loaded they are more likely to use emotion in decision making. Fourth, a participant's behavior (e.g. drinking more of one product) can be influenced by multiple things, including affective liking and stressful autonomic arousal as two possibilities; the ability here to measure and combine more than one mode allows to better disambiguate the cause of a behavior (e.g. discount the amount of product consumed due to liking by considering what part of the consumption was due to stress). Finally, a participant's prediction is influenced by the immediate affective feeling state experienced at the time of making a decision (e.g., incidental mood states such as happy, angry, sad, anxious, or energetic state).

In an exemplary implementation of this invention, repeated random-outcome trials are used to assess consumer preferences, as follows: In each trial, a participant is asked to select one of two beverage dispensers. Each beverage dispenser randomly dispenses one beverage (Product 1) some times and another beverage (Product 2) some times. The probability that Product 1 will be dispensed is higher for one beverage dispenser than the other dispenser. The participant is not told the probability for either dispenser. The participant selects a dispenser, and a beverage is dispensed from it into a cup. Each cup is numbered or otherwise labeled, to show whether it holds Product 1 or Product 2. Thus, the participant can tell, by looking at the cup, whether Product 1 or Product 2 has been dispensed from the selected dispenser. The participant sips the beverage. The participant then self-reports an evaluation of the product, by answering one or more questions relating to the product. Repeated trials are done, each starting with the participant being asked to select a beverage dispenser.

A participant who wants to taste a particular beverage (e.g., Product 1) has to guess which beverage dispenser will dispense that product. Repeated trials may enable a participant to determine which of the two dispensers is most likely to dispense the desired beverage.

In this example, a participant's facial valence (positive, neutral, or negative affect expressed by facial or head movements or lack of such movement) may be observed multiple times during each trial, including: (a) after a participant learns the outcome of his or her selection (e.g., when the participant learns which beverage has been dispensed), but before the participant tastes the beverage, (b) while a participant is tasting a beverage and shortly thereafter, and (c) during the period that a person gives a report related to said tasting experience. The latter time period is of interest because the face or head movements may “leak” feelings while the person is otherwise concentrating on answering the questionnaire. For example, a person's head may shake and lips curl when encountering a bad aftertaste, and the person may forget to suppress this socially inappropriate display while engaged with the computer's questions. In this example, facial valence is categorized as positive (satisfied), neutral or negative (dissatisfied).

Gathering data on affect at different times during a taste test is highly advantageous, because a person may display different affect at different points during the test. For example, a person's affect may be different immediately after tasting a product than while giving a cognitive self-report on the experience. Interestingly, the very act of reporting may change affect about an experience. Also, for example, a person may be inhibited from expressing disgust after sipping a distasteful beverage (e.g., because of social training that it is impolite to stick out one's tongue after tasting something bad.) But the same person may feel free to express intense negative affect if a machine dispenses a beverage that he or she does not want.

Repetitive random-outcome tasks, such as the beverage tasting tasks described above, have two strong advantages. First, they make people consume both products (e.g., sip both beverages) randomly, even in the later trials. As a result, people are more likely to have multiple encounters with each product (e.g., multiple tastes of both beverages) and are more likely to find what they really like. This is important, because a person's preference for a product may change after repeated experiences. For example, in beverage taste tests, people often prefer a sweeter beverage on their first sip, although they don't like the beverage eventually. This indicates that people often require long-term multiple experiences of a product that is mildly bad before they actually notice its badness.

Second, participants' desire (or wanting) of different outcomes can be inferred by means of their affective responses to obtained outcomes. When participants choose an option that they predict is more likely to give the outcome that they want, and then actually obtain their desired outcome, they tend to show positive facial valence (or satisfaction response). But when they obtain the outcome they don't want, they tend to show negative facial valence (or disappointment response). For example, if a participant chooses a beverage dispensing machine (option) that the participant predicts is more likely to dispense a desired beverage product (outcome), and then actually obtains the beverage product (outcome) that the participant wants, the participant tends to show a satisfied facial expression.

In an exemplary implementation of this invention, affective wanting (e.g., how much a person wants to have a product in the future, as distinguished from how much a person liked prior experiences of the product) may be measured at different times, depending on the type of measurement. For example, the strength of wanting may be inferred from electrodermal activity before making a selection or while awaiting an expected outcome. Or, for example, the positive (or negative) nature of the wanting may be inferred after the fact, by observing facial valence immediately after a person receives an outcome of a selection (e.g., receives a beverage cup marked to show which beverage it holds). If a person responds with positive facial valence to Product 1 being dispensed, this increases the probability that the person wanted Product 1 before it was dispensed; if a person responds with negative facial valence, then it probably was not wanted. The combination of multiple measures gives a stronger indication of wanting, e.g., if the product anticipation period is accompanied by high skin conductance (or by another measure of sympathetic nervous system activity), and if the facial valence upon receiving the product is negative, then the combination suggests the product was strongly not wanted, and perhaps even dreaded.

This invention may be implemented in such a manner that participants repeatedly perform tasks and at least five types of data are gathered that shed light on their preferences regarding a product. These five types of data are cognitive liking metrics, cognitive wanting metrics, affective liking metrics, affective wanting metrics and behavioral data.

First, some definitions:

“Product” means a product or service. Packaging or advertising may itself be a “product”.

“Random” means random or pseudo-random. “Randomly” means randomly or pseudo-randomly.

The terms “report” and “self-report” (and grammatical variations thereof) each refer to feedback given by one or more persons, which feedback requires cognitive processing by that person or persons. Consider the following example: A person is asked “How much do you like or dislike this product?”, and answers “very much”. The answer in this example is a “report”, the answer is “reported” and the person is “reporting”. The answer can be given in any way, for example, by selecting the words “very much” on a computer screen.

A “cognitive wanting metric” is a numerical value that is indicative of at least one reported want or reported desire of at least one person regarding at least one future (as of the time of the report) experience relating to a product. Consider the following example: A person sips a sample of a beverage product. The person is asked “If the beverage you just tasted was available where you usually shop, which of the following best describes how likely you would be to buy it?”, and is given a choice of five answers. These possible answers are assigned numerical values, e.g., ranging from 1 for “definitely would not buy it” to 5 for “definitely would buy it”. If the person selects one of these answers, the numerical value associated with that answer is an example of a “cognitive wanting metric” regarding the product.

A “cognitive liking metric” is a numerical value (other than a cognitive wanting metric) that is indicative of at least one person's reported feelings about a product. Consider the following example: A person sips a sample of a beverage product. The person is asked “how much do you like or dislike your current sip?”, and is given a choice of nine answers. The possible answers are assigned numerical values, ranging from 1 for “dislike it extremely” to 9 for “like it extremely”. Each of these numerical values corresponding to these answers is an example of a “cognitive liking metric”.

An “affective wanting metric” is a numerical value that is indicative of the state of at least one physical or physiologic aspect of a person during either (1) a period starting up to ten seconds before a person makes a selection of at least one of a plurality of sources (e.g., one of two beverage dispensing machines) that dispense or otherwise provide products, and ending when such selection is made, or (2) a three second period starting when a person learns the outcome of such a selection (e.g., when a person learns which beverage is dispensed from a beverage dispensing machine selected by said person). Consider the following example: Immediately after a person learns which beverage product has been dispensed from a beverage dispensing machine selected by that person (e.g., by looking at a cup that is numbered in such a manner as to indicate which beverage product it holds), the person's facial valence is observed. The facial valence is classified as positive (e.g., satisfied), neutral, or negative (dissatisfied). These possible facial valences are assigned the numerical values of 1, 0 and −1, respectively. In this example, the numerical value of that facial valence is an “affective wanting metric”. Another example: a numerical value indicative of a participant's average skin conductance during the ten seconds immediately before the participant makes such a selection is an “affective wanting metric”. This metric is of particular interest when the participant fully expects to receive a particular outcome, e.g. when the uncertainty associated with the random trials is removed, and the participant is awaiting the product they are told they will receive.

An “affective liking metric” is a numerical value that is indicative of the state of at least one physical or physiologic aspect of a person, during the period starting with, and including, when a person samples a product and ending with, and including, when that person first reports about that sample. Consider the following example: A person tastes a beverage by sipping it and then pauses before reporting about that sip. Immediately after the sip, the person's facial valence is observed. The facial valence is classified as positive (e.g., satisfied), neutral, or negative (dissatisfied). These possible facial valences are assigned the numerical values of 1, 0 and −1, respectively. In this example, the numerical value of that facial valence is an “affective liking metric”.

This invention may be implemented as a method comprising the following steps, in combination: (a) exposing one or more persons to one or more products, or generating instructions relating to said exposure, (b) using one or more sensors to measure one or more physical or physiologic parameters regarding at least some of said persons, which data comprises at least one affective wanting metric with respect to at least one said product, (c) using a processor to calculate, based at least in part on said data comprising said affective wanting metric, at least one numerical value with respect to at least one preference, attitude or feeling regarding at least one said product. Furthermore: (1) at least some said data may be gathered in trials with random outcomes, (2) at least one said affective wanting metric may be sympathetic nervous system response measured by contact with the skin, (3) at least one said affective wanting metric may be sympathetic nervous system arousal, (4) at least one said affective wanting metric may be electrodermal activity, and (5) at least one said affective wanting metric may be facial valence. Said calculation of said numerical value may be based at least in part on data comprising an affective liking metric, in which case data indicative of a physical or physiologic parameter regarding at least some of said persons may be accepted, which data comprises said affective liking metric. Said calculation of said numerical value may be based at least in part on data comprising a cognitive liking metric or a cognitive wanting metric, in which case data indicative of at least one report by at least one said person may be accepted, which said report is indicative of said cognitive wanting metric or said cognitive liking metric.

This invention may be implemented as a method comprising the following steps in combination, any one or more of which steps may be performed one or more times: (a) using an input device to receive data indicative of a person's selection of at least one of a plurality of alternatives, (b) exposing said person to an outcome comprised of all or part of a product, in such a manner that the probability that said person will be exposed to said outcome is less than 100% and is dependent on which selection said person makes, or generating instructions relating to said exposure, (c) using a sensor or other apparatus to obtain data indicative of the state of at least one physical or physiological parameter of said person during or within thirty seconds before or after said exposure, and (d) using at least one processor to calculate, based at least in part on data regarding one or more of said selections and said states, at least one numerical value relating to at least one preference, attitude or feeling with respect to at least one product. Furthermore: (1) said data may be gathered from more than one person and processed by said processor or processors, (2) each said person may make multiple selections, (3) at least one said parameter may be facial valence, (4) at least one said parameter may be electrodermal activity, (5) at least one said parameter may be a measure of sympathetic nervous system arousal, (6) said processor may be adapted to process data indicative of reports inputted by a participant interacting with a graphical user interface displayed by a computer screen, and (7) one or more other steps may be added, in which the participant knows, before making a selection of a source of a product, that said source has a 100% probability of dispensing a particular product.

This invention may be implemented as computer instructions in machine-readable format for using one or more processors to perform the following steps, in combination: (a) accepting data indicative of a person's selection of at least one of a plurality of alternatives, (b) generating instructions regarding exposing said person to an outcome comprised of a product, in such a manner that the probability that said person will be exposed to said outcome is less than 100% and is dependent on which selection said person makes, (c) accepting data indicative of at least one report of said person regarding at least one of said outcome or said alternative, (d) accepting data obtained by at least one sensor, which data is indicative of the state of at least one physical parameter of said person during or within ten seconds before or after said exposure, and (e) calculating, based on at least some of said data regarding one or more of said selections, reports or states, a numerical value indicative of at least one preference, attitude or feeling relating to at least one product. Furthermore: (1) said instructions may be adapted for accepting data for multiple trials, (2) at least one said sensor may be a video camera, (3) at least one said sensor may measure electrodermal activity, and (4) one or more said processors may accept data with respect to more than one persons, regarding one or more of said selections, reports or states, and may calculate, based on at least some of such data with respect to said more than one persons, a numerical value indicative of at least one preference, attitude or feeling relating to at least one product.

This invention may be implemented as an apparatus comprising, in combination: (a) sensors for measuring one or more physical or physiologic variables regarding at least some of said persons, which data comprises at least one affective wanting metric and at least one affective liking metric, and (b) at least one processor for calculating, based at least in part on said data, at least one numerical value with respect to at least one preference, attitude or feeling regarding at least one said product. Furthermore: (1) at least some said data may be measured during tasks in which a participant makes at least one selection with a random outcome, and (2) at least some said data may be measured during multiple exposures of a participant to a product.

### BRIEF DESCRIPTION OF THE DRAWINGS

In the detailed description which follows, reference will be made to the attached drawings.

FIG. 1 is a flow chart illustrating a participant's decision making task in repetitive random outcome trials, in an implementation of this invention.

FIG. 2 is a timeline illustrating the sequence for each trial, in an implementation of this invention.

FIG. 3 is a timeline illustrating when data is collected in each trial, in an implementation of this invention.

FIG. 4 shows a setup used for repetitive random outcome trials involving a beverage product, in an implementation of this invention.

FIG. 5 shows a user interface, displayed before choosing an option, in an implementation of this invention.

FIG. 6 shows a user interface, displayed after selecting an option and obtaining an outcome, in an implementation of this invention.

FIG. 7 shows a user interface displayed in every trial, which asks for a self-report about liking or disliking a sip of a product, in an implementation of this invention.

FIG. 8 shows a user interface displaying three questions, the bottom two of which are displayed every fifth trial, in an implementation of this invention.

FIG. 9 shows another user interface displaying three questions, the bottom two of which are displayed every fifth trial, in an implementation of this invention.

FIG. 10 is a flowchart illustrating affective measures, in an implementation of this invention.

FIG. 11 is a flowchart illustrating cognitive measures, in an implementation of this invention.

FIG. 12 is a flowchart illustrating the computation of affective, cognitive, and affective-cognitive values, in an implementation of this invention.

FIG. 13 is a flowchart illustrating computational analysis over participants, in an implementation of this invention.

### DETAILED DESCRIPTION

In an exemplary implementation of this invention, repeated random-outcome trials are used to assess consumer preferences, as follows: In each trial, a participant is asked to select one of two beverage dispensers. Each beverage dispenser randomly dispenses one beverage (Product 1) some times and another beverage (Product 2) some times. The probability that Product 1 will be dispensed is higher for one beverage dispenser than the other dispenser. The participant is not told the probability for either dispenser. The participant selects a dispenser, and a beverage is dispensed from it into a cup. Each cup is numbered or otherwise labeled, to show whether it holds Product 1 or Product 2. Thus, the participant can tell, by looking at the cup, whether Product 1 or Product 2 has been dispensed from the selected dispenser. The participant sips the beverage. The participant then self-reports an evaluation of the product, by answering one or more questions relating to the product. Repeated trials are done, each starting with the participant being asked to select a beverage dispenser.

A participant who wants to taste a particular beverage (e.g., Product 1) has to guess which beverage dispenser will dispense that product. Repeated trials may enable a participant to determine which of the two dispensers is most likely to dispense the desired beverage.

According to the principles of this invention, decision-making tasks and user interfaces (UI) may be used for eliciting participants' expressions of their feelings about a product. Thus, the expression of customer feelings is less likely to be influenced by a human facilitator or observer, and the influence of the user interface can be controlled across the conditions being compared.

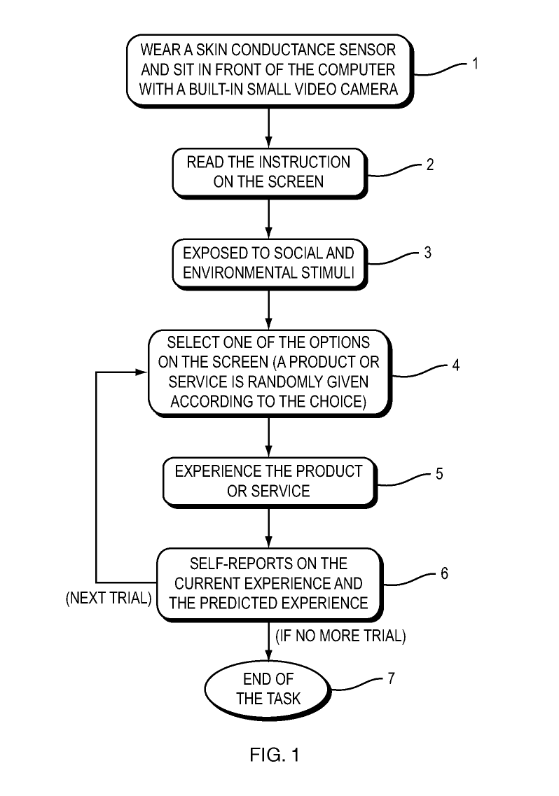

FIG. 1 illustrates the steps of a participant's decision-making task in repetitive random outcome trials, in an exemplary implementation of this invention. In the first step 1, the participant wears an electrodermal activity sensor and sits in front of a computer screen with a built-in small video camera. The participant continues to wear the sensor and to sit in front of the screen throughout the remainder of the task. In the second step 2, the participant reads the instruction on the computer screen. In an optional third step 3, the participant may be exposed to some social and environmental stimuli. In the fourth step 4, the participant selects one of the options displayed on the computer screen, and a product is randomly provided according to the selection. In the fifth step 5, the participant experiences the product. In the sixth step 6, the participant self-reports on the current experience and the predicted experience. After this sixth step, the next trial starts at step 4 until a certain number of trials have been performed. At that point, after the sixth step, the task is done 7.

According to principles of this invention, multiple decision-making trials may be conducted. In each trial, participants make a selection among two or more options, and obtain an outcome (i.e., a sample product or service) for the selected option. Each option has a unique outcome distribution over different products.

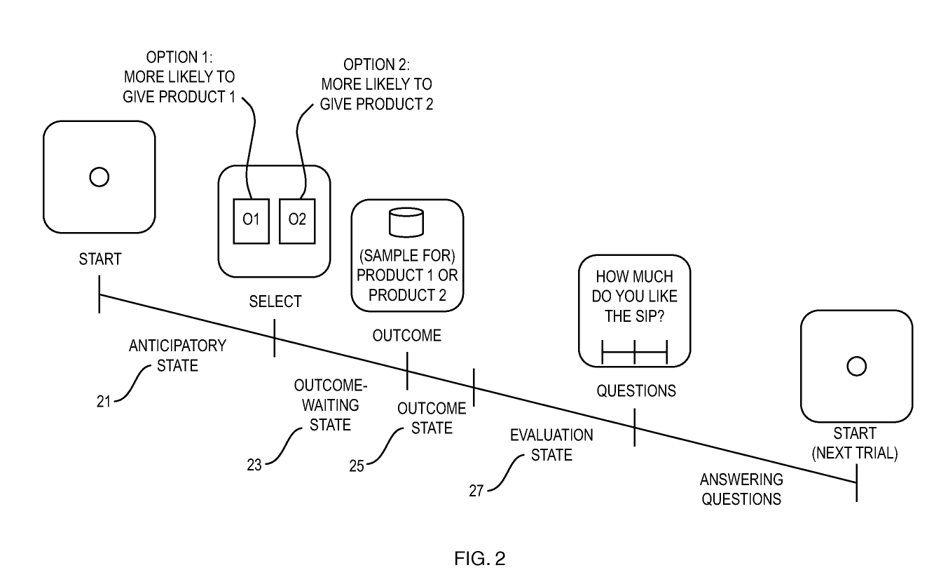

FIG. 2 illustrates the sequence of each trial, in an exemplary implementation of this invention. As shown in FIG. 2, in each trial, participants experience an anticipatory state 21 before and while choosing an option, an outcome-waiting state 23 before knowing an outcome, an outcome state 25 right after obtaining an outcome, and an evaluation state 27 while evaluating the obtained outcome.

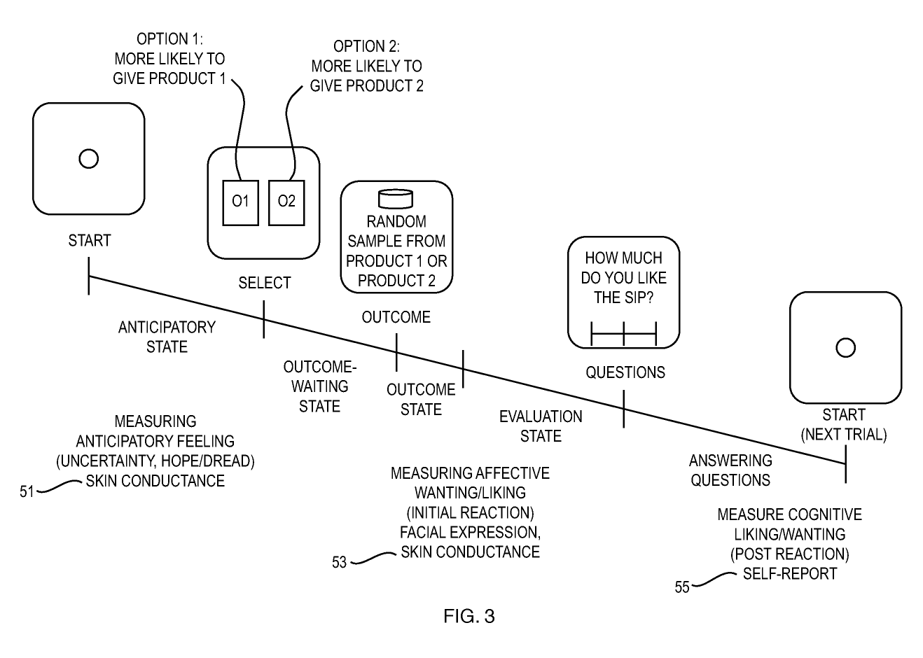

FIG. 3 illustrates types of data that may be collected, in an exemplary implementation of this invention. In the anticipatory state, data indicative of anticipatory feeling 51 may be gathered. For example, electrodermal activity may be measured at this time. In the outcome state, data indicative of a participant's affect (e.g., wanting or liking) 53 may be gathered. For example, facial expression and electrodermal activity may be measured at this time. In the evaluation state, including answering questions, data indicative of a participant's cognitive liking and wanting 55 may be gathered.

Each participant's decision making may take the form of a repetitive random-outcome (gambling-like) task where such participant has multiple trials to make a selection among several options and each option selection provides a random outcome according to its designed outcome probability distribution. For instance, in the case of two available options (Option 1 and Option 2) and two available outcomes (P1 (a sample for Product 1) and P2 (a sample for Product 2), this invention may be implemented in such a way that Option 1 is more likely to provide outcome P1 than outcome P2 and Option 2 is more likely to provide outcome P2 than outcome P1. Participants initially don't know about the underlying outcome probability distribution of each option. Participants' goal may be to figure out which option more often provides the outcome they enjoy more and select that option more often. Thus, participants tend to select the option that is more likely to provide their favored outcome more often.

For example, this invention may be implemented with a beverage preference test regarding Pepsi Vanilla® and Pepsi Summer Mix®.

Before this beverage preference test, each participant is given the following instruction:
“On your computer, there are two vending machines, Machine 1 (left side) and Machine 2 (right side). Each vending machine will direct you to take a sip of flavored cola, either Pepsi Vanilla® (beverage 135), or Pepsi Summer Mix® (beverage 246). One vending machine may be or may not be more likely to provide you with more opportunity to taste beverage 135 and the other with 246.
In addition to tasting beverages and answering questions, your goal will be to figure out which machine more often directs you to drink the beverage you enjoy more and select that machine more often.
You will be asked to make multiple machine selections and please choose freely between the two vending machines.”

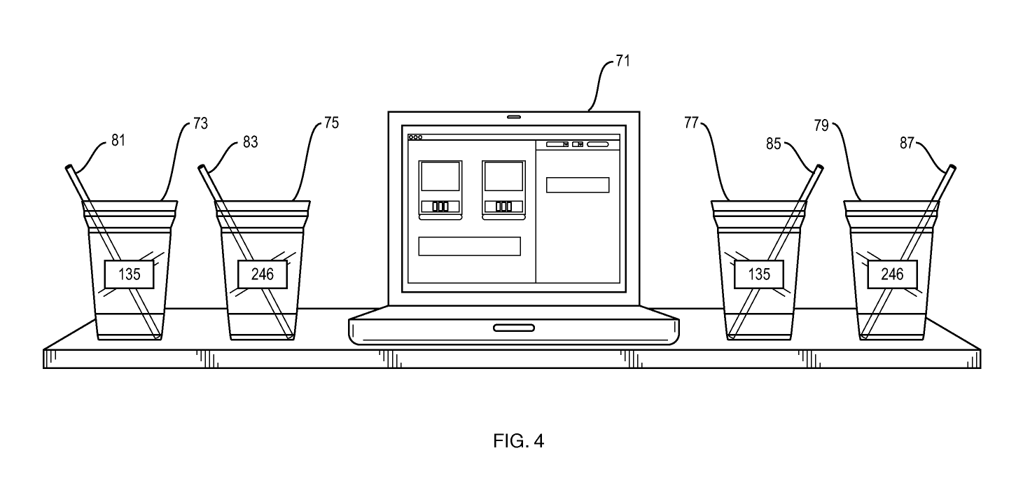

FIG. 4 shows the general setup of this beverage preference test. A computer screen 71 provides a graphical user interface. Four cups are provided, for participant's use in the tasting beverage samples. Two of the cups 73, 77 are labeled 135, which label is indicative that the cup is for holding beverage 135. Two of the cups 75, 79 are labeled 246, which label is indicative that the cup is for holding beverage 246. Any beverage in cups 73 and 75 is dispensed from Machine 1. Any beverage in cups 77 and 79 is dispensed from Machine 2. Straws 81, 83, 85, 87 are used for avoiding blocking a participant's facial expressions. Each participant has 30 trials of selection and each trial is composed of the steps in FIG. 4. Eventually participants were expected to realize that each machine favors a different product, and select the vending machine hoping to receive their favored product.

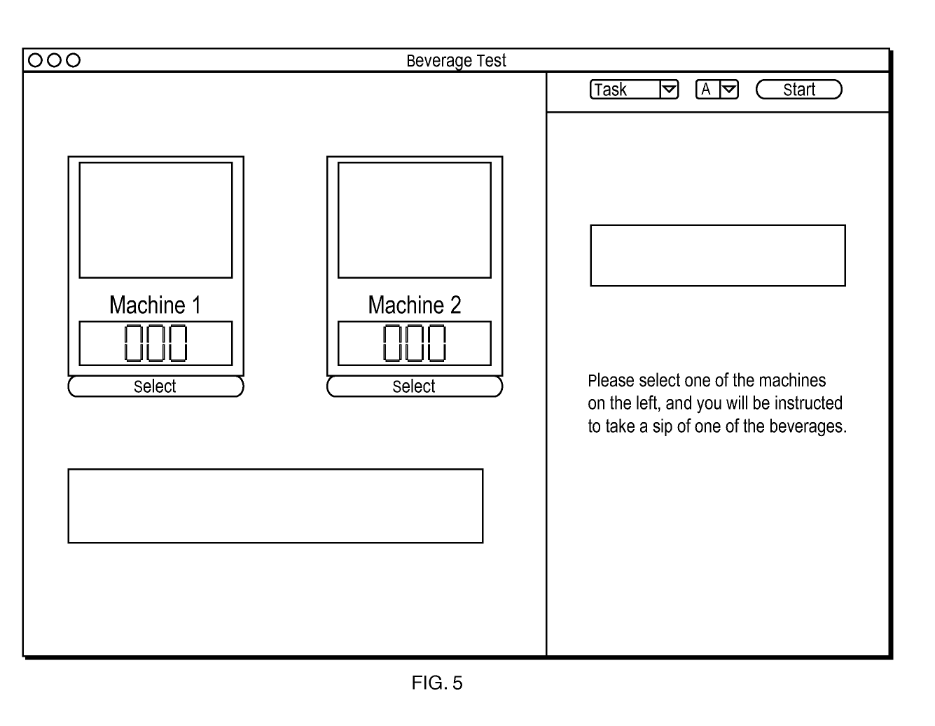

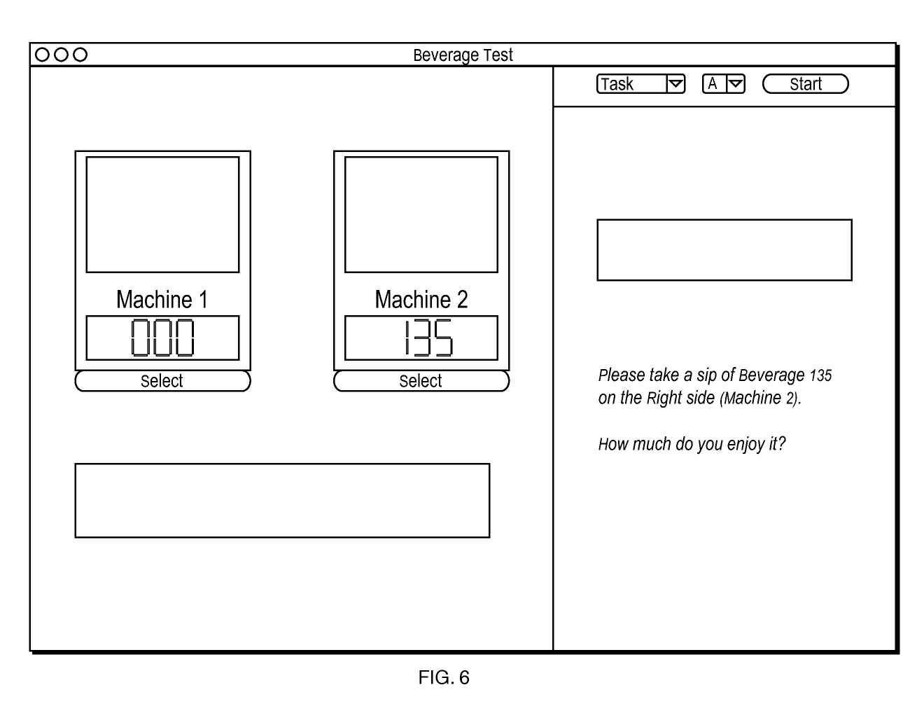

FIG. 5 shows the screen before choosing an option (Machine 1 or Machine 2) in each trial. FIG. 6 shows the screen after selecting an option (Machine 2) and obtaining an outcome (Pepsi Vanilla®, 135) for the selected option.

In this example, Machine 1 provides Pepsi Vanilla® 70% of the time and Pepsi Summer Mix® 30% of the time; whereas Machine 2 provides Pepsi Vanilla® 30% of the time and Pepsi Summer Mix® 70% of the time. Also, for half of the participants, Machine 1 is on the left side and Machine 2 is on the right side. For the other half of the participants, Machine 2 is on the left side and Machine 1 is on the right side.

These repetitive random-outcome tasks have two strong advantages. First, they make people sip both beverages randomly, even in the later trials. As a result, people are more likely to have multiple tastes of both beverages and are more likely to find what they really like. This is important, because a person's preference for a product may change after repeated experiences. For example, in beverage taste tests, people often prefer a sweeter beverage on their first sip, although they don't like the beverage eventually. This indicates that people often require long-term multiple experiences of a product that is mildly bad before they actually notice its badness.

Second, participants' desire (or wanting) of different outcomes can be inferred by means of their affective responses to obtained outcomes. For instance, when participants have chosen the option they predict is more likely to give the outcome they want and actually obtain their desired outcome, they may show positive facial valence (or satisfaction response). But when they obtain the outcome they don't want, they may show negative facial valence (or disappointment response).

Affective states caused by any social events (e.g., economic crisis, terrorist attacks, or professional sporting events) or advertisements (e.g., brand images or any visual cues) may influence customers' preference for products and services. Thus, in order to test how these different factors change customers' overall preference, social and environmental factors can be incorporated into decision-making tasks in order to manipulate participants' choice conditions. For example, as shown in FIG. 1, a participant may be exposed to social and environmental stimuli 3 before selecting an option on the screen 4. These stimuli may also be used to relax or neutralize a participant's mood before sampling a product.

According to principles of this invention, a multi-modal approach may be used, rather than cognitive measures only. The multi-modal approach may include behavioral measures (e.g., physical number of choices, amount consumed), affective measures (e.g. sensor measures of facial expression) and cognitive measures (e.g., self-reports).

This multi-modal measurement can provide more robust assessment of participants' preferences than cognitive measures alone because of the following main reasons: First, the human brain uses both emotion (affect) and cognition in decision-making and evaluation processes. Second, participants in an experiment are likely to cognitively bias their self-report of what they like. Third, when people are cognitively loaded they are more likely to use emotion in decision making Fourth, a participant's behavior (e.g. drinking more of one product) can be influenced by multiple things, including affective liking and stressful autonomic arousal as two possibilities; the ability here to measure and combine more than one mode allows to better disambiguate the cause of a behavior (e.g. discount the amount of product consumed due to liking by considering what part of the consumption was due to stress). Finally, a participant's prediction is influenced by the immediate affective feeling state experienced at the time of making a decision (e.g., incidental mood states such as happy, angry, sad, anxious, or energetic state).

In an exemplary implementation of this invention, the following multi-modal measurements are taken: electrodermal activity, facial valence and cognitive measures (in the form of self-reports).

In this exemplary implementation, skin conductance is measured in three time windows in each trial: (1) after a person makes a selection and while he or she anticipates receiving a product (to detect the average level of a participant's anticipatory arousal), (2) right after a participant samples a product (to detect the average level of outcome arousal), and (3) during the evaluation of the outcome (to detect the average level of evaluation arousal). A skin conductance baseline measure is also computed from one or more relaxation periods, during which the electrodermal activity signal is low and slowly changing. These relaxation periods may occur, for example, at the start of a task, during an optional exposure to environmental stimuli, or at the end of a task.

Also, in this exemplary implementation, facial expression is detected with a video camera during multiple time windows in each trial, e.g.: (1) right after participants obtain their selection outcome (in the outcome state), and (2) during the participant's evaluation of the outcome (in the evaluation state). When participants chose the option they predict is more likely to give the outcome they want and actually obtain their desired outcome, they tend to show positive facial valence (or satisfaction response). But when they obtain the outcome they don't want, they tend to show negative facial valence (or disappointment response). The term “FV” refers to facial valence, i.e., the positive, neutral or negative feelings expressed by facial or head movements or lack of such movement (such as positive if a smile, nod or lip licking, or negative if a frown, nose wrinkle, tongue protrusion, or head shake.). The terms “outcome facial valence” and “outcome FV” refer to facial valence during the outcome state. The terms “evaluation facial valence” and “evaluation FV” refer to facial valence during the evaluation state, including during the period in which the participants answer questions.

Also, in this exemplary implementation, cognitive questions are used (such as self-reports on “how much do you like this product” and “how likely are you to buy this product”.) There are two kinds of questions. One kind are trial-based questions (asked in all or some trial), and the other kind are after-the-test questions (asked once after all the trials). After-the-test questions measure participants' after-the-test preference for products.

The following beverage preference test is an example of a multi-modal approach in accordance with the principles of this invention. Participants are asked to wear an electrodermal activity sensor on the non-dominant hand, and to sit in front of a computer that includes a small camera taking video of their facial-head movements. They then follow the instructions on the computer, which ask them to make a choice between two machines and take a sip of the outcome beverage and answer questions on the computer. Each participant has 30 trials of this choice-sipping-question process in the experiment. Participants' choice and sipping behavioral information (behavioral measure), facial expression and electrodermal activity information (affective measure) and self-reports on questions (cognitive measure) are recorded.

In this example, FV is coded as follows: Two persons look at each video and code outcome FV and evaluation FV shown in that video. Outcome and evaluation FVs are coded as positive (1)/negative (−1)/neutral (0) responses. Positive FV comprises smiling, nodding, looking pleased, satisfied, or licking the lips. Negative FV comprises frowning, shaking the head, showing disgust, or otherwise looking displeased or disappointed. If the two persons disagree on the coding of a FV, then the FV is categorized as neutral. Alternately, the coding may be done by a computer using automated software for analyzing facial expressions together with head movements.

In this example, screen shots are displayed at different steps during each trial. FIGS. 5, 6, 7, 8 and 9 illustrate these screen shots. They show user interfaces for giving instructions, displaying questions and obtaining the participants' answers (self-reports).

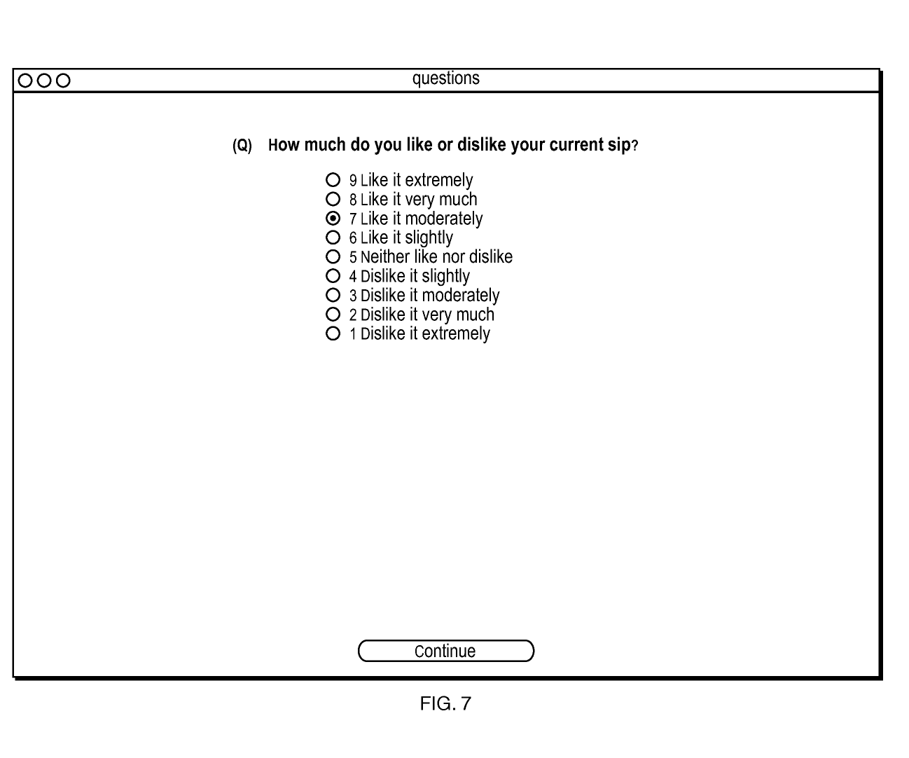

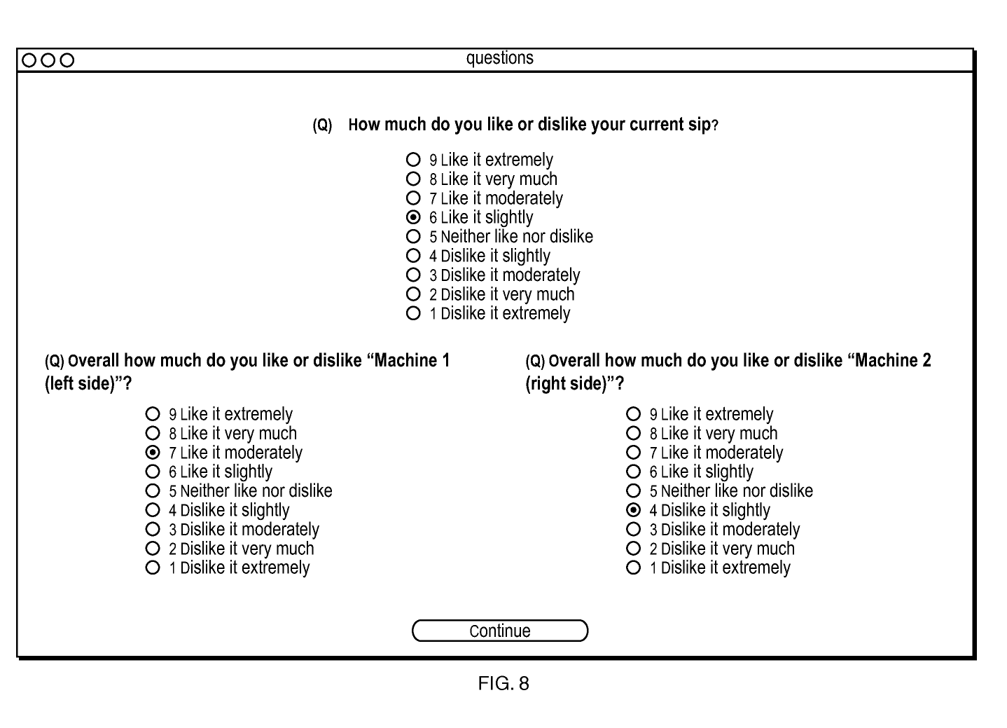

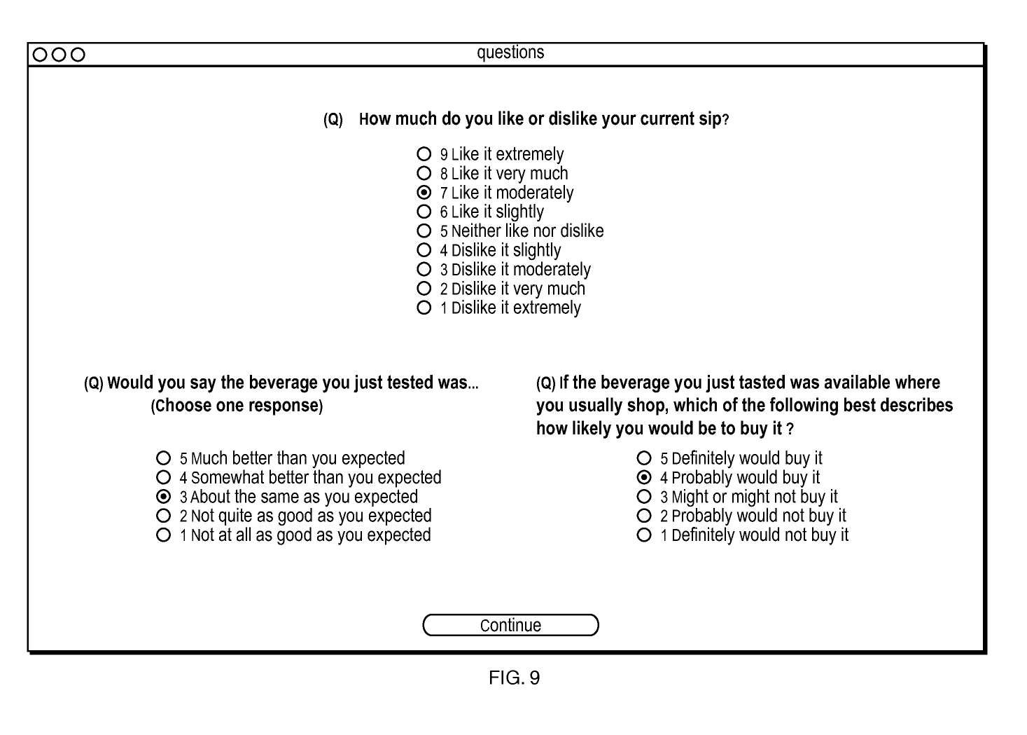

In this example, the question for a participant's beverage liking (in FIG. 7) is asked every trial. In the (5n−1)th trial (n=1, . . . , 6), two questions for a participant's machine liking (in FIG. 8) are asked additionally to the beverage-liking question. Also, in the (5n) th trial (n=1, . . . , 6), two questions about a participant's expectation comparison and purchase intent (in FIG. 9) are also asked.

In this example, the two questions (in FIG. 8), “Overall how much do you like or dislike the Machine 1 (or 2)?” are asked to obtain cognitive wanting values (i.e., the expected pleasure of the future outcome of options before making a choice). In this example, the question, “How much do you like or dislike your current sip?” is used to obtain the cognitive liking value. The answers are scaled to −4 to 4 as in the parentheses as follows: Like it extremely (=4), Like it very much (=3), Like it moderately (=2), Like it slightly (=1), Neither like nor dislike (=0), Dislike it slightly (=−1), Dislike it moderately (=−2), Dislike it very much (=−3), Dislike it extremely (=−4).

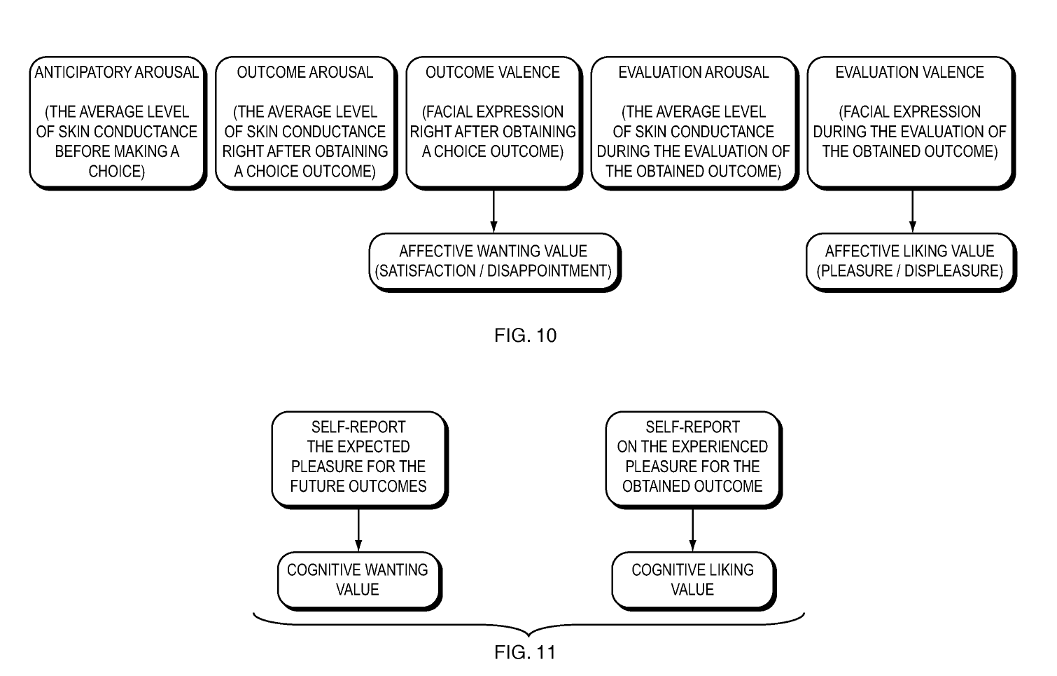

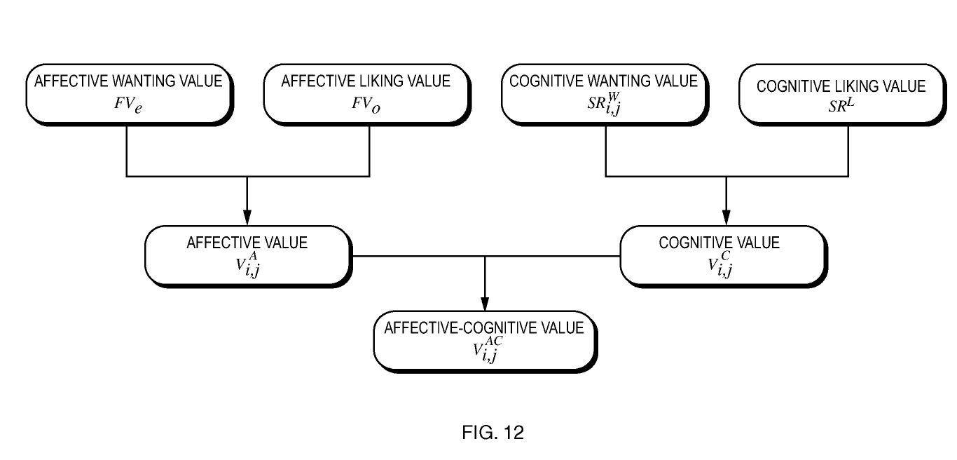

FIGS. 10 and 11 are flowcharts that illustrate examples of data collection, according to principles of this invention. FIG. 10 shows measures of affect (such as anticipatory arousal, outcome arousal, outcome valence, evaluation arousal and evaluation valence) that may be obtained, and indicates that some of these measures yield affective wanting and affective liking values. FIG. 11 shows that cognitive liking and cognitive wanting values may be obtained from self-reports.

Behavioral measures may be taken in addition to affective and cognitive measures, according to principles of this invention. These behavioral measures include the participants' machine choices and corresponding outcomes (e.g., which product dispenser a person chooses and which product is dispensed) as well as the sipped amount of each beverage through each machine during the task. The amount sipped may be determined, for example, by comparing the amount of beverage dispensed with the amount remaining in the cup after the task is completed.

In an exemplary implementation of this invention, the following computational model is applied to infer participants' preferences for products. This model analyzes the participants' behavioral information and information on participants' affective and cognitive wanting and liking during a repetitive random-outcome task.

For this computational model, the following notations are used:
V_{i,j}^{A}(t): affective value for option i, outcome j at trial t
V_{i,j}^{C}(t): cognitive value for option i, outcome j at trial t
V_{i,j}^{AC}(t): affective-cognitive value for option i, outcome j at trial t
c(t): option chosen at trial t, i.e., c(t)=i means selecting option i (=1, . . . , I)
p(t): outcome (product) sampled at trial t, i.e.,
   p(t)=j means obtaining outcome j (=1, . . . , J)
S: total number of participants
I: total number of options (e.g., the options may be two beverage dispensing machines that may be selected)
M: total number of products (outcomes)
T: total number of trials in the task
FVₒ(t): affective liking value, outcome FV at trial t
FVₑ(t): affective wanting value, evaluation FV at trial t
SR_{i,j}^{W}(t): cognitive wanting value, self-reported (SR) value on the expected pleasure/displeasure of future outcome j with option i at trial t (answered before making a choice)
SR^{L} (t): cognitive liking value, self-reported (SR) value on the pleasure/displeasure of the obtained outcome after making a choice at trial t

In this computational model:

$${FV}_{o} ( t ) = \{ \begin{matrix}1 & if outcome FV at trial t is positive ( i . e . , satisfaction ) \\ 0 & if outcome FV at trial t is neutral \\ - 1 & if outcome FV at trial t is negative ( i . e . , disappointment )\end{matrix} \\ {FV}_{s} ( t ) = \{ \begin{matrix}1 & if sip FV at trial t is positive ( i . e . , liking ) \\ 0 & if sip FV at trial t is neutral \\ - 1 & if sip FV at trial t is negative ( i . e . , disliking )\end{matrix}$$

FV(t)=wₒFVₒ(t)+wₑ FVₑ(t) where wₒ and wₑ (≧0) are weights given on outcome FV and evaluation FV each. For example, equal weighting may be employed: wₒ=wₑ=0.5

When the participant selects option c(t) and obtains outcome p(t) at trial t(=1, . . . , T), affective value V_{i,j}^{A}(t) is defined as:

$${V}_{i , j}^{A} ( t ) = \{ \begin{matrix}1 & if i = c ( t ) , j = p ( t ) and FV ( t ) > 0 \\ - 1 & if i = c ( t ) , j = p ( t ) and FV ( t ) < 0 \\ 0 & otherwise\end{matrix}$$

Cognitive measures are obtained, such as participants' self-reports (SR) on the expected pleasure of the future outcome of each option before making a choice. These cognitive measures may include SR_{i,j}^{W}(t) (self-reported wanting for all i and j pairs at trial t) and SR^{L}(t) (level of enjoyment after evaluating the obtained outcome at trial t). Thus, SR_{i,j}^{W}(t) and SR^{L}(t) are defined by the questions for self-reports in each trial. For example, the SR values can be scaled to −4 to 4 as follows: Like it extremely (=4), Like it very much (=3), Like it moderately (=2), Like it slightly (=1), Neither like nor dislike (=0), Dislike it slightly (=−1), Dislike it moderately (=−2), Dislike it very much (=−3), Dislike it extremely (=−4). (Note that positive values mean liking and negative values mean disliking in the scale.)

When the participant selects option c(t) and obtains outcome p(t) at trial t(=1, . . . , T), cognitive value V_{i,j}^{C}(t) is defined as:

$${V}_{i , j}^{C} ( t ) = \{ \begin{matrix}{w}_{1} {SR}_{i , j}^{W} ( t ) + {w}_{2} {SR}^{L} ( t ) & if i = c ( t ) , j = p ( t ) \\ {w}_{1} {SR}_{i , j}^{W} ( t ) & otherwise\end{matrix}$$

where w₁+w₂=1, w₁≧0, w₂≧0.

In this computational model, in an illustrative beverage preference test, w₁=0, w₂=1. That is,

$${V}_{i , j}^{C} ( t ) = \{ \begin{matrix}{SR}^{L} ( t ) & if i = c ( t ) , j = p ( t ) \\ 0 & otherwise .\end{matrix}$$

When the participant selects option c(t) and obtains outcome p(t) at trial t(=1, . . . , T), the choice-outcome matrix for each trial t is defined as follows:

$${C}_{i , j} ( t ) = \{ \begin{matrix}1 & if i = c ( t ) , j = p ( t ) \\ 0 & otherwise\end{matrix}$$

Now the total number of that option i is selected over all the trials is:

$${N}^{I} ( i ) = {\sum }_{t = 1}^{T} {\sum }_{j = 1}^{J} {C}_{i , j} ( t )$$

Also, the total number of times that outcome j is obtained over all the trials is:

$${N}^{J} ( j ) = {\sum }_{t = 1}^{T} {\sum }_{j = 1}^{I} {C}_{i , j} ( t )$$

For each outcome (product) j, the total numbers of positive or negative affective values over all the participants, M₊^{A}(j) and M₋^{A}(j), are defined as follows:

Vi,j^{A}(t|s)≡Vi,j^{A}(t) for participant s=(1, . . . , S).

$${M}_{+}^{A} ( j ) = {\sum }_{s = 1}^{S} {\sum }_{t = 1}^{T} {\sum }_{i = 1}^{I} pos [ {V}_{i , j}^{A} ( t \vert s ) ] where pos ( x ) = \{ \begin{matrix}1 & if x > 0 \\ 0 & otherwise\end{matrix} \\ {M}_{-}^{A} ( j ) = {\sum }_{s = 1}^{S} {\sum }_{t = 1}^{T} {\sum }_{i = 1}^{I} neg [ {V}_{i , j}^{A} ( t \vert s ) ] where neg ( x ) = \{ \begin{matrix}1 & if x < 0 \\ 0 & otherwise\end{matrix}$$

Similarly, the total number of positive or negative cognitive values over all the participants, M₊^{C}(j) and M₋^{C}(j), respectively, is defined as.

Vi,j^{X}(t|s)≡Vi,j^{C}(t) for participant s=(1, . . . , S).

$${M}_{+}^{C} ( j ) = {\sum }_{s = 1}^{S} {\sum }_{t = 1}^{T} {\sum }_{i = 1}^{I} pos [ {V}_{i , j}^{C} ( t \vert s ) ] where pos ( x ) = \{ \begin{matrix}1 & if x > 0 \\ 0 & otherwise\end{matrix} \\ {M}_{-}^{C} ( j ) = {\sum }_{s = 1}^{S} {\sum }_{t = 1}^{T} {\sum }_{i = 1}^{I} neg [ {V}_{i , j}^{C} ( t \vert s ) ] where neg ( x ) = \{ \begin{matrix}1 & if x < 0 \\ 0 & otherwise\end{matrix}$$

The affective-cognitive value is defined as:

$${V}_{i , j}^{A C} ( t ) = \{ \begin{matrix}1 & if {V}_{i , j}^{A} ( t ) > 0 and {V}_{i , j}^{C} ( t ) > 0 \\ - 1 & if {V}_{i , j}^{A} ( t ) < 0 and {V}_{i , j}^{C} ( t ) < 0 \\ 0 & otherwise\end{matrix}$$

(Note that this definition disregards the cases where the affective value conflicts with the cognitive value.)

Now we define the total number of positive or negative affective-cognitive values over all the participants, M₊^{AC}(j) and M₋^{AC}(j), respectively.

Vi,j^{AC}(t|s)≡V_{i,j}^{AC}(t) for participant s=(1, . . . , S).

$${M}_{+}^{A C} ( j ) = {\sum }_{s = 1}^{S} {\sum }_{t = 1}^{T} {\sum }_{i = 1}^{I} pos [ {V}_{i , j}^{A C} ( t \vert s ) ] where pos ( x ) = \{ \begin{matrix}1 & if x > 0 \\ 0 & otherwise\end{matrix} \\ {M}_{-}^{A C} ( j ) = {\sum }_{s = 1}^{S} {\sum }_{t = 1}^{T} {\sum }_{i = 1}^{I} neg [ {V}_{i , j}^{A C} ( t \vert s ) ] where neg ( x ) = \{ \begin{matrix}1 & if x < 0 \\ 0 & otherwise\end{matrix}$$

Two different products (j=1,2) can be compared in terms of several criteria.

For product 1, consider three different kinds of pairs {M₊^{X}(1), M₋^{X}(1)} from three different models X=(A, C, or AC). Similarly, for product 2, consider {M₊^{X}(2), M₋^{X}(2)} X=(A, C, or AC).

For a model X=(A, C, or AC), assuming M₊^{X}(1)≧M₊^{X}(2), the following quantities may be computed, in accordance with principles of this invention:

Sensitivity=M₊^{X}(1)/(M₊^{X}(1)+M₋^{X}(1))

Specificity=M₋^{X}(2)/(M₊^{X}(2)+M₋^{X}(2))

Likelihood ratio positive (LR+)=Sensitivity/(1−Specificity)

Likelihood ratio negative (LR−)=(1−Sensitivity)/Specificity

Accuracy=(M₊^{X}(1)+M₋^{X}(2))/(M₊^{X}(1)+M₋^{X}(1)+M₊^{X}(2)+M₋^{X}(2))

MCC (Matthews correlation coefficient)=

$$\frac{{M}_{+}^{X} ( 1 ) {M}_{-}^{X} ( 2 ) - {M}_{+}^{X} ( 2 ) {M}_{-}^{X} ( 1 )}{\sqrt{( {M}_{+}^{X} ( 1 ) + {M}_{+}^{X} ( 2 ) ) ( {M}_{-}^{X} ( 1 ) + {M}_{-}^{X} ( 2 ) ) ( {M}_{+}^{X} ( 1 ) + {M}_{-}^{X} ( 1 ) ) ( {M}_{+}^{X} ( 2 ) + {M}_{-}^{X} ( 2 ) )}}$$

If a certain model X=(A, C, or AC) tends to provide higher LR+, lower LR−, higher accuracy and higher MCC than other models, then that model X reflects the difference in customers' preferences of two products more clearly than other models, in terms of these criteria (LR+, LR−, Accuracy, MCC).

For example, in a beverage preference test according to principles of this invention, the AC model provides provide higher LR+, lower LR−, higher accuracy and higher MCC than A and C models.

For each participant s, the average cognitive value for product j over trials is computed as follows:

$${μ}_{j}^{C} ( s ) = \frac{{\sum }_{i = 1}^{I} [ {V}_{i , j}^{C} ( t \vert s ) ]}{{N}^{J} ( j \vert s )} where {N}^{J} ( j \vert s ) = {\sum }_{t = 1}^{T} {\sum }_{i = 1}^{I} {C}_{i , j} ( t \vert s )$$

is the total number of obtaining product j over trials. Here, C_{i j}(t|s)=C_{i j}(t) for participant s=(1, . . . , S).

The mean of μᵢ^{C}(s) over all the participants is computed as:

$$〈 {μ}_{j}^{C} ( s ) 〉 = ( 1 / S ) {\sum }_{s = 1}^{S} {μ}_{j}^{C} ( s ) .$$

Now, for each participant s, the average cognitive value difference for products 1 and 2 is defined as: Diff₁₋₂^{C}(s)=μ₁^{C}(s)−μ₂^{C}(s).

Thus, the mean of average cognitive value differences for products 1 and 2 over all the participants are computed as follows:

$$〈 {Diff}_{1 - 2}^{C} ( s ) 〉 = ( 1 / S ) {\sum }_{s = 1}^{S} {Diff}_{1 - 2}^{C} ( s ) = 〈 {μ}_{1}^{C} ( s ) 〉 - 〈 {μ}_{2}^{C} ( s ) 〉 .$$

When [symbol] Diff₁₋₂^{C}(s) [symbol] >0, participants prefer product 1 to product 2 in terms of the mean of average cognitive values.

For each participant s, the weighted affective value cⱼ^{A}(s) for product is defined as: cⱼ^{A}(s)=w₊n₊^{A}(s)−w₋n₋^{A}(s) where n₊^{A}(s) and n₋^{A}(s) are the number of positive and negative affective values over all the trials, respectively, and w₊≧0 and w₋≦0. Note that

$${n}_{+}^{A} ( s ) ≡ {\sum }_{t = 1}^{T} {\sum }_{i = 1}^{I} pos [ {V}_{i , j}^{A} ( t \vert s ) ] and$$

$${n}_{-}^{A} ( s ) ≡ {\sum }_{t = 1}^{T} {\sum }_{i = 1}^{I} neg [ {V}_{i , j}^{A} ( t \vert s ) ]$$

In beverage preference tests, it may be advantageous to put four times more weight on negative affective values (or facial valences) than on positive affective values (i.e., w₊=0.5 and w₋=2) may be advantageous, since such weighting may best explain the participants' after-the-test preference in terms of the weighted affective values. Weights may be learned by seeing which values best predict marketplace or other behavioral outcomes or by using learned values from products that are similar to those being tested.

The mean of cⱼ^{A} (s) over all the participants is computed as:

$$〈 {c}_{j}^{A} ( s ) 〉 = ( 1 / S ) {\sum }_{s = 1}^{S} {c}_{j}^{A} ( s ) .$$

For each participant s, the weighted affective value difference for products 1 and 2 is defined as: Diff₁₋₂^{A}(s)=c₁^{A}(s)−c₂^{A}(s).

Thus, the mean of weighted affective value differences for products 1 and 2 over all the participants are computed as follows:

$$〈 {Diff}_{1 - 2}^{A} ( s ) 〉 = ( 1 / S ) {\sum }_{s = 1}^{S} {Diff}_{1 - 2}^{A} ( s ) = 〈 {c}_{1}^{A} ( s ) 〉 - 〈 {c}_{2}^{A} ( s ) 〉 .$$

When [symbol] Diff₁₋₂^{A}(s) [symbol] >0, participants prefer product 1 over product 2, in terms of the mean of weighted affective values.

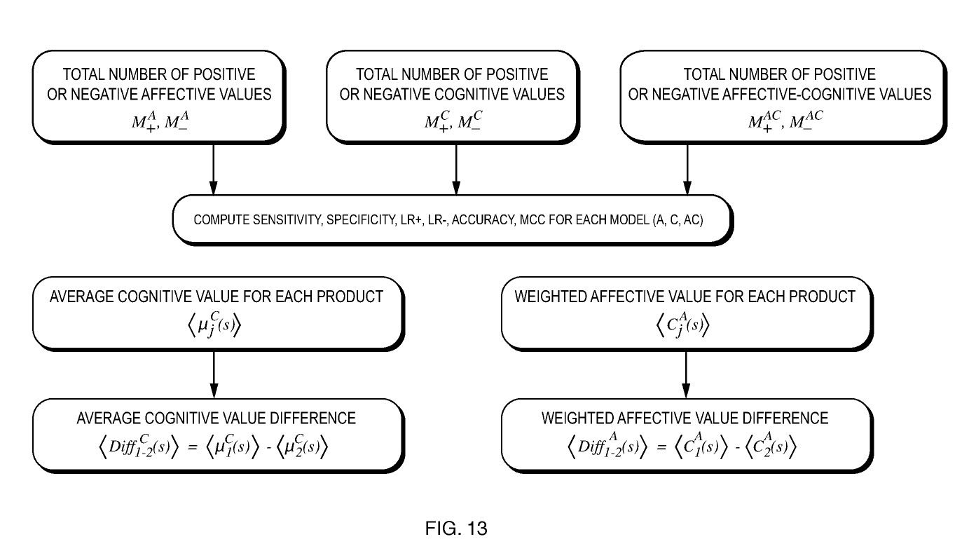

FIG. 13 and FIG. 14 summarize an example of computational analysis used in this invention. FIG. 13 shows how the affective, cognitive and affective-cognitive values are computed for each participant. FIG. 14 shows the computational analysis over all the participants to compute the mean of average cognitive value differences and the mean of weighted affective value differences.

The participants may be divided into two groups: the FV expressive group and the FV non-expressive group. For example, the FV expressive group may be defined to be the group of participants who showed any outcome or evaluation FV's at least four times over all the 30 trials. This invention may be implemented in such a manner that only FV data with respect to the FV expressive group is analyzed. Other cognitive and affective measures may still be used for the group without expressive FV.

It is advantageous to keep the following in mind, when analyzing data in implementations of this invention:

When random-outcome trials are used, people's self-reported liking values and behavior may change with the uncertainty of the situation they are in. For example, when participants pick a machine that has a high probability of giving their favorite beverage, they tend to report higher liking after the sip of their favorite beverage than when there is a lot of uncertainty before getting their favorite beverage. In other words, more uncertainty (or arousal, which is a main component of stress and surprise) may result in lower liking ratings compared to sipping the same beverage received under a highly certain condition. According to principles of this invention, this condition may be disambiguated by measuring not only how much product is consumed but also skin conductance (which is indicative of arousal or stress that may influence consumption).

Also, in some cases, the average size of the sips that people take is larger when they obtain an unexpected outcome (e.g., having more arousal, which can also occur when surprised or when stressed). The larger sips may happen regardless of whether it was the person's preferred beverage or not.

Also, electrodermal activity tends to increases with uncertainty. Choosing a more unpredictable machine may be associated with higher skin conductance than choosing a more predictable machine. There tends to be more physiological arousal when there is more uncertainty, and this internal arousal can modulate what a person feels, thinks, decides, and does. Skin conductance is one of several measures that can be used to provide a real-time continuous measure of this arousal component of affect.

Decision scientists in different fields such as psychology, neuroscience and economics have been trying to understand how humans make decisions and judgments and build a unified theory of decision-making. Current important findings suggest that humans do not use a single decision-making mechanism such as in the expected utility (EU) theory in modern economics, even for a simple decision-making task such as a choice between two lotteries. Rather, multiple valuation systems such as cognitive and affective processing systematically influence human decision-making. Also, the neural substrates of liking (pleasure) are separate from those of wanting (motivation) in the human brain, so there is evidence from neuroscience that supports treating these concepts differently when modeling how people make decisions.

Most attempts to date that try to predict marketplace decisions are based on studies where people are asked what they would do, i.e. on self-report data. Self-reports on experienced utility captures cognitive elements of liking (what you think you can say that you like) but may not capture the wanting, desire or motivation of purchasing. It is also interesting to note that while self-reported liking can be rated instantly, obtaining an accurate value for wanting may require a longer experience.

This invention has a clear advantage over these prior approaches, because it may be implemented in such a way as to measure participants' anticipatory feeling, self-reported cognitive wanting/liking and sensor-measured affective wanting/liking during decision-making and evaluation processes in order to describe their choice and predict future market outcomes of new products.

An exemplary multi-modal implementation of this invention is very helpful for analyzing the cases where there is disagreement between self-reported liking and facial expressions. For instance, such a multi-modal approach can detect when implicit liking or disliking appears as affective arousal or facial expressions without cognitive knowing or cognitive feeling (explicit liking or disliking).). It can also detect when a physiological expression of arousal or facial valence occurs sooner during the trials than self-reported feelings; thus, it may provide accurate information with fewer trials than does measuring a person's self-report over time.

This invention may be applied to many different industries that need a mechanism to predict future marketplace outcomes of new products. For example, this invention is useful for industries where customers' affective feeling state (e.g., anticipatory, visceral, mood state) has significant influence on their decision-making. Also, for example, this invention is useful when the customers' affective liking state (i.e. pleasure/displeasure as revealed on facial expressions) is more likely to be revealed when appreciating products.

This invention may be implemented in ways other than the embodiments described above. The following are some examples of alternative implementations:

Alternatively, this invention may be applied to a single-trial task and a deterministic-outcome task.

Alternatively, (i) different computational models may be used to combine affective, cognitive and behavioral measures, (ii) different cognitive, affective and behavioral measures may be employed, and (iii) different products may be tested, including products other than beverages.

Also, although products were dispensed into cups labeled with numbers in one example above, numbers need not be used as labels. Rather, labels can be adapted to whatever is appropriate for the product being tested.

Alternatively, packaging itself may the focus of the testing, or the packaging and how it interacts with the product may be the focus of the testing.

Alternatively, products other than beverages may be tested. Even if beverages are tested, the manner of implementation of the beverage test may be different than as described above. For example, straws may not be used and the number of trials may be different. Also, for example, in different instantiations of this invention, specific questions and their timing may be adapted to the needs of a product test so that they capture the aspects of the human-product interaction being evaluated. Likewise, for example, a participant need not sit, e.g., if sitting is not an ordinary position for experiencing the product. This invention can be used regardless of the participant's position. Similarly, multiple trials may be conducted in which participants are in different positions or contexts, and data from these multiple trials may be combined.

Electrodermal activity is at this time the most common measure of sympathetic nervous system arousal and is typically measured by passing a very tiny current through electrodes placed on the surface of the skin and recording either conductance or resistance; alternatively it may be measured simply by a voltage potential. Alternately, sympathetic nervous system arousal may be measured without contact with the skin. For example it might be observed in changes in pupil dilation or other information that can be gathered using a camera (video or other) pointed at the face. This invention may be implemented with any other measure of sympathetic nervous system arousal.

### CONCLUSION

It is to be understood that the methods and apparatus which have been described above are merely illustrative applications of the principles of the invention. Numerous modifications may be made by those skilled in the art without departing from the scope of the invention. The scope of this invention is limited only by the claims that follow.

What is claimed is:

1. A method comprising the following steps, in combination:
exposing one or more persons to one or more products, or generating instructions relating to said exposure,
using one or more sensors to measure one or more physical or physiologic parameters regarding at least some of said persons, which data comprises at least one affective wanting metric with respect to at least one said product,
using a processor to calculate, based at least in part on said data comprising said affective wanting metric, at least one numerical value with respect to at least one preference, attitude or feeling regarding at least one said product.

2. A method as set forth in claim 1, in which at least some said data is gathered in trials with random outcomes.

3. A method as set forth in claim 1, in which in which at least some said data is gathered in multiple trials.

4. A method as set forth in claim 1, in which at least one said affective wanting metric is a measure of sympathetic nervous system arousal.

5. A method as set forth in claim 1, in which at least one said affective wanting metric is a measure of electrodermal activity.

6. A method as set forth in claim 1, in which at least one said affective wanting metric is facial valence.

7. A method as set forth in claim 1, wherein said calculation of said numerical value is based at least in part on data comprising an affective liking metric, and wherein said method further comprises the step of accepting data indicative of a physical or physiologic parameter regarding at least some of said persons, which data comprises said affective liking metric.

8. A method as set forth in claim 1, wherein said calculation of said numerical value is based at least in part on data comprising a cognitive liking metric or a cognitive wanting metric, and wherein said method further comprises the step of accepting data indicative of at least one report by at least one said person, which said report is indicative of said cognitive wanting metric or said cognitive liking metric.

9. A method comprising the following steps in combination, any one or more of which steps may be performed one or more times:
using an input device to receive data indicative of a person's selection of at least one of a plurality of alternatives,
exposing said person to an outcome comprised of all or part of a product, in such a manner that the probability that said person will be exposed to said outcome is less than 100% and is dependent on which selection said person makes, or generating instructions relating to said exposure,
using at least one sensor to obtain data indicative of the state of at least one physical or physiological parameter of said person during or within thirty seconds before or after said exposure, and
using at least one processor to calculate, based at least in part on data regarding one or more of said selections and said states, at least one numerical value relating to at least one preference, attitude or feeling with respect to at least one product.

10. A method as set forth in claim 9, in which data is gathered from more than one person and processed by said processor or processors.

11. A method as set forth in claim 9, in which said person makes multiple selections.

12. A method as set forth in claim 9, in which at least one said parameter is facial valence.

13. A method as set forth in claim 9, in which at least one said parameter is electrodermal activity.

14. A method as set forth in claim 9, in which at least one said parameter is a measure of sympathetic nervous system arousal.

15. A method as set forth in claim 9, in which said processor is adapted to process data indicative of reports inputted by a participant interacting with a graphical user interface displayed by a computer screen.

16. A method as set forth in claim 9, further comprising at least one step in which said participant knows, before making a selection of a source of a product, that said source has a 100% probability of dispensing a particular product.

17. Computer instructions in machine-readable format for using one or more processors to perform the following steps, in combination:
accepting data indicative of a person's selection of at least one of a plurality of alternatives,
generating instructions regarding exposing said person to an outcome comprised of a product, in such a manner that the probability that said person will be exposed to said outcome is less than 100% and is dependent on which selection said person makes,
accepting data indicative of at least one report of said person regarding at least one of said outcome or said alternative,
accepting data obtained by a sensor or other apparatus, which data is indicative of the state of at least one physical parameter of said person during or within ten seconds before or after said exposure, and
calculating, based on at least some of said data regarding one or more of said selections, reports or states, a numerical value indicative of at least one preference, attitude or feeling relating to at least one product.

18. Computer instructions as set forth in claim 17, in which said instructions are adapted for accepting data for multiple trials.

19. Computer instructions as set forth in claim 17, in which at least one said sensor is a video camera.

20. Computer instructions as set forth in claim 17, in which at least one said sensor measures skin conductance.

21. Computer instructions as set forth in claim 17, in which said one or more processors accept data with respect to more than one persons, regarding one or more of said selections, reports or states, and calculates, based on at least some of such data with respect to said more than one persons, a numerical value indicative of at least one preference, attitude or feeling relating to at least one product.

22. Apparatus comprising, in combination:
sensors for measuring one or more physical or physiologic variables regarding at least some of said persons, which data comprises at least one affective wanting metric and at least one affective liking metric, and
at least one processor for calculating, based at least in part on said data, at least one numerical value with respect to at least one preference, attitude or feeling regarding at least one said product.

23. Apparatus as set forth in claim 22, adapted for measuring at least some said data during tasks in which a participant makes at least one selection with a random outcome.

24. Apparatus as set forth in claim 22, adapted for measuring at least some said data during multiple exposures of a participant to a product.
